(19) United States
(12) Patent Application Publication
(10) Pub. No.: US 2004/0064843 A1
(43) Pub. Date: Apr. 1, 2004

(54) Process for estimating random error in chemical and biological assays

(76) Inventors: Edward Susko, Halifax (CA); Robert Nadon, Baie D'Urfe (CA)

Correspondence Address: Darby & Darby, P O Box 5257, New York, NY, 10150-5257, US

(21) Appl. No.: 10/363,727

(22) PCT Filed: Sep. 7, 2001
(86) PCT No.: PCT/IB01/01625

(51) Int. Cl. A01K 67/027

(52) U.S. Cl. 800/15

(57) ABSTRACT

A method is disclosed for improving the reliability of physical measurements obtained from array hybridization studies performed on an array having a large number of genomic samples including a replicate subset containing a small number of replicates insufficient for making precise and valid statistical inferences. An error in measurement of a sample is estimated by combining estimates obtained with individual samples in the replicate subset, and utilizing the estimated sample error as a standard for accepting or rejecting the measurement of a sample under test.

### FIELD OF THE INVENTION

The present invention relates to a process for improving the accuracy and reliability of physical experiments performed on hybridization arrays used for chemical and biological assays. In accordance with the present invention, this is achieved by estimating the extent of random error present in replicate samples constituting a small number of data points from a statistical point of view.

### BACKGROUND OF THE INVENTION

Array-based genetic analyses start with a large library of cDNAs or oligonucleotides (robes), immobilized on a substrate. The probes are hybridized with a single labeled sequence, or a labeled complex mixture derived from a tissue or cell line messenger RNA (target). As used herein, the term “probe” will therefore be understood to refer to material tethered to the array, and the term “target” will refer to material that is applied to the probes on the array, so that hybridization may occur.

The term “element” will refer to a spot on an array. Array elements reflect probe/target interactions. The term “background” will refer to area on the substrate outside of the elements.

The term “replicates” will refer to two or more measured values of the same probe/target interaction. Replicates may be within arrays, across arrays, within experiments, across experiments, or any combination thereof.

Measured values of probe/target interactions are a function of their true values and of measurement error. The term “outlier” will refer to an extreme value in a distribution of values. Outlier data often result from uncorrectable measurement errors and are typically deleted from further statistical analysis.

There are two kinds of error, random and systematic, which affect the extent to which observed (measured) values deviate from their true values. Random errors produce fluctuations in observed values of the same process or attribute. The extent and the distributional form of random errors can be detected by repeated measurements of the same process or attribute. Low random error corresponds to high precision. Systematic errors produce shifts (offsets) in measured values. Measured values with systematic errors are said to be “biased”. Systematic errors cannot be detected by repeated measurements of the same process or attribute because the bias affects the repeated measurements equally. Low systematic error corresponds to high accuracy. The terms “systematic error”, “bias”, and “offset” will be used inter-changeably in the present document.

An invention for estimating random error present in replicate genomic samples composed of small numbers of data points has been described by Ramm and Nadon in “Process for Evaluating Chemical and Biological Assays”, International Application No. PCT/IB99/00734, filed Apr. 22, 1999, the entire disclosure of which is incorporated herein by reference. In a preferred embodiment, the process described therein assumed that, prior to conducting statistical tests, systematic error in the measurements had been removed and that outliers had been deleted.

Once systematic error has been removed, any remaining measurement error is, in theory, random. Random error reflects the expected statistical variation in a measured value. A measured value may consist, for example, of a single value, a summary of values (mean, median), a difference between single or summary values, or a difference between differences. In order for two values to be considered significantly different from each other, their difference must exceed a threshold defined jointly by the measurement error associated with the difference and by a specified probability of concluding erroneously that the two values differ (Type I error rate). Statistical tests are conducted to determine if values differ significantly from each other.

In addition, to correct removal of systematic error, many statistical tests require the assumption that residuals be normally distributed. When it is incorrectly assumed that residuals are normally distributed, the calculation of the residuals and of subsequent statistical tests is biased. Residuals reflect the difference between values' estimated true scores and their observed (measured) scores. If a residual score is extreme (relative to other scores in the distribution), it is called an outlier. An outlier is typically removed from further statistical analysis because it generally indicates that the measured value contains excessive measurement error that cannot be corrected. In order to achieve normally distributed residuals, data transformation is often necessary (e.g., log transform). Two approaches have been presented in prior art.

Piétu et al. (1996) observed in their study that a histogram of probe intensities presented a bimodal distribution. They observed further that the distribution of smaller values appeared to follow a Gaussian distribution. In a manner not described in their publication, they “fitted” the distribution of smaller values to a Gaussian curve and used a threshold of 1.96 standard deviations above the mean of the Gaussian curve to distinguish nonsignals (smaller than the threshold) from signals (larger than the threshold). Based on calculation of residuals, the present invention also provides for threshold estimations. However, the present invention differs from Piétu et al. (1996) in that it:

uses replicates;

uses formal statistical methods to obtain threshold values;

does not assume a Gaussian (or any other) distribution; and

can detect outlier values.

Chen, Dougherty, & Bittner have presented an analytical mathematical approach that estimates the distribution of non-replicated differential ratios under the null hypothesis. This approach is similar to the present invention in that it derives a method for obtaining confidence intervals and probability estimates for differences in probe intensities across different conditions. It differs from the present invention in how it obtains these estimates. Unlike the present invention, the Chen et al. approach does not obtain measurement error estimates from replicate probe values. Instead, the measurement error associated with ratios of probe intensities between conditions is obtained via mathematical derivation of the null hypothesis distribution of ratios. That is, Chen et al. derive what the distribution of ratios would be if none of the probes showed differences in measured values across conditions that were greater than would be expected by “chance.” Based on this derivation, they establish thresholds for statistically reliable ratios of probe intensities across two conditions. The method, as derived, assumes that most genes do not show a treatment effect and that the measurement error associated with probe intensities is normally distributed (i.e., that the Treatment/Reference ratios are normally distributed around a ratio of approximately 1). The method, as derived, cannot accommodate other measurement error models (e.g., lognormal). It also assumes that all measured values are unbiased and reliable estimates of the “true” probe intensity. That is, it is assumed that none of the probe intensities are “outlier” values that should be excluded from analysis. Indeed, outlier detection is not possible with the approach described by Chen et al. The present invention differs from Chen et al. (1997) in that it:

uses replicates

does not assume a Gaussian (or any other) distribution;

can be applied to single condition data (i.e., does not require 2 conditions to form ratios);

does not require the assumption that most genes do not show a treatment effect; and

can detect outliers.

In accordance with one aspect, the present invention is a process for estimating the extent of random error present in replicate genomic samples composed of small numbers of data points and for conducting a statistical test comparing expression level across conditions (e.g., diseased versus normal tissue). It is an alternative to the method described by Ramm and Nadon in “Process for Evaluating Chemical and Biological Assays”, International Application No. PCT/IB99/00734. As such, it can be used in addition to (or in place of) the procedures described by Ramm and Nadon. In accordance with another aspect, the present invention is a process for establishing thresholds within a single distribution of expression values obtained from one condition or from an arithmetic operation of values from two conditions (e.g., ratios, differences). It is an alternative to the deconvolution process described in International Application No. PCT/IB99/00734. In accordance with a third aspect, it is a process for detecting and deleting outliers.

### BRIEF DESCRIPTION OF THE DRAWINGS

The foregoing brief description, a well as further objects, features and advantages of the present invention will be understood more completely from the following detailed description of a presently preferred, but nonetheless illustrative embodiment, with reference being had to the accompanying drawings, in which:

FIG. 1 shows the results of residual estimation based on simulated data; and

FIGS. 2 and 3 shows results of residual estimation based on actual experimental data.

### DESCRIPTION OF THE PREFERRED EMBODIMENT

We assume throughout that we observe data yᵢⱼ, with i=1, . . . , n and j=1, . . . , in where:

Yᵢⱼ=μᵢ+εᵢⱼ (1)

and the εᵢⱼ are independent and identically distributed. Our interest is in estimating the residual distribution, the distribution of the εᵢⱼ. Let ƒ,ƒ* and F denote the density, characteristic function and cumulative distribution function of the εᵢⱼ.

A tacit assumption is that n is large and m is small, for instance 2 or 3. Assumptions such as these arise naturally in measurement error models. While our interest in estimating the residual distribution arose in the analysis of gene expression data, we expect the methodology to be of broader applicability.

With m moderate to large, the usual estimate of the residual distribution is a discrete distribution that gives equal mass to each of the estimated residuals:

$${\hat{F}}_{e} ( e ) = \frac{1}{nm} {\sum }_{i = 1}^{n} {\sum }_{j = 1}^{m} I \{ {y}_{ij} - {\overline{y}}_{i} \geq ϵ \} \qquad ( 2 )$$

This estimator is biased with the bias dependent up n the residual distribution. For instance, for a N (0 ,1) residual distribution the expectation of \hat{F}ₑ is the N (0,(m−1)/m ) distribution. For a Cauchy residual distribution, the expectation is the distribution of a Cauchy random variable multiplied by 2−2/m. When the residual distribution has finite mean, the bias decreases with increasing m. With n large and m small however, the bias dominates the variance. In contrast the methods presented here give consistent (large n ) estimates of the residual distribution. The basic idea uses the differences in observations, yᵢⱼ₁−yᵢⱼ₂ which have distributions that depend, in a known way, upon the residual distribution alone. This differs from the usual way of calculating residuals. An example best illustrates this difference. Consider the three replicate values of 1, 2, and 3. The usual way of calculating residuals is to subtract the mean of the three values from each value in turn (1-2; 2-2; 3-2) yielding three residuals (−1, 0, 1). In the preferred form of the present process, the residuals are calculated instead by subtracting each replicate value from each of the other replicate values in all possible permutations. In the present example, this would be (Replicate 1-Replicate 2; Replicate 2-Replicate 1; Replicate 1-Replicate 3; Replicate 3-Replicate 1; Replicate 2-Replicate 3; Replicate 3-Replicate 2), that is, (1-2; 2-1; 1-3; 3-1;2-3; 3-2) to yield six residuals (-1,1, -2,2,-1,1). This approach has the advantage of not including the potentially biasing effect of including the mean in the calculations. Alternatively, all possible combinations (rather than permutations) might be used.

Two methodologies are proposed: inversion of an estimate of the characteristic function of the residuals and an E-M algorithm approach that seeks a residual distribution that maximizes a pseudo-likelihood for the differenced data. A key reference for the characteristic function methodology is Zhang (1990). Background material for the E-M algorithm is available in Dempster, Laird and Rubin (1977) and McLachlan and Krishnan (1997).

### Array Based Expression Data

The estimation of residual distributions became of interest to us in the analysis of array based gene expression intensity data. Regardless of the technology used (macroarrays, microarrays, or biochips) or the labeling method (radio-is topic, fluorescent, or multi-fluorescent), the observed values reflect the total amount of hybridization (joining) of two complementary strands of DNA to form a double-stranded molecule. The log-transformed observations (radio-isotopic, fluorescent, fluorescent ratios) can be labeled y_{gij} where g denotes the experimental condition that the observed values correspond to (for instance, drug versus control, different tissues, etc.).

The index i indicates the genetic sequence tag used in the experiment and j indicates that the observation was the jth repeated measurement within the genetic sequence tag/condition. The model for the y_{gij} is:

Y_{gij}=μ_{gi}+σₛε_{gij}

where the ε_{gij} are assumed independent and identically distributed. Here the ε_{gij} are measurement errors; μ_{gi} is the true intensity value for the gth condition and ith tag. Primary interest is in μ₁ᵢ−μ₂ᵢ the difference in the intensity values, for a given genetic sequence tag, between two different conditions. A gene's expression intensity reflects its activity at specific moments or circumstances according to the design of the study. A gene's activity is of interest in its own right and also because it usually reflects the production of protein, which has corollaries for the function and regulation of cells, tissues, and organs in the body. Differences in gene expression are of interest to the extent that they reflect differences across conditions of these biological processes. Gene expression data have been characterized by large measurement error variation, large numbers of comparisons (sequence tags) and small numbers of measurements for each sequence tag. The number of comparisons can range between a few hundred and hundreds of thousands. The numbers of measurements for a given sequence tag and condition are often 2 or 3. Because the measurement error is non-negligible it is usually the case that confidence intervals for the differences μ₁ᵢ−μ₂ᵢ are desired. One approach is to make the common assumption that the residuals are normally distributed, in which case (1−α)×100% confidence intervals would be provided by

\overline{y}₁ᵢ−\overline{y}₂ᵢ±z_{α/2}{square root over ((σ₁²/m+σ₂²/m)}

Here σ_{g}² is the measurement error variance for the gth condition. With known non-normal residual distributions different forms of confidence intervals would usually be considered but it would still be reasonable to consider intervals with center \overline{y}₁ᵢ−\overline{y}₂ᵢ and half-width a constant multiple τ of \sqrt{σ₁²/m+σ₂²/m}. What value of τ to use depends upon the particular form of the residual distribution. For the normal distribution τ is z_{α/2}, for the double exponential exponential distribution it would be −log(α). Thus, for instance, to obtain a 95% confidence interval τ=1.96 would be used for a normal residual distribution and τ=3 would be used for the double exponential. These very different values of τ indicate that the residual distribution for a given condition is important t the inferences of interest in the analysis of expression data. Because of the similarities in the measurement process across comparisons and the large number of comparisons, it should be possible to obtain estimates of the residual distribution with low variability. Because of the small number of measurements for each comparison, care has to be taken to avoid bias in estimation.

### Characteristic Function Methodology

One approach to estimation of the residual distribution is through the characteristic function for the Yᵢⱼ₁−Yᵢⱼ₂. Since Yᵢⱼ₁−Yᵢⱼ₂=ε_{ij1-ε}ᵢⱼ₂ this characteristic function is ƒ*(t)ƒ*(−t). The form of the characteristic function for the difference indicates several identifiability problems. If the residual distribution is not a symmetric distribution then the distribution of −εᵢⱼ is not the same as the distribution of εᵢⱼ. However, since the characteristic function of −εᵢⱼ is ƒ*(−t), the characteristic function for the difference εᵢⱼ−εᵢⱼ₂ is ƒ*(t)ƒ*(−t) whether the residual distribution is that of −εᵢⱼ or εᵢⱼ. Thus skewness in the residual distribution will not be recoverable from the distribution of the difference of two errors. A common assumption for measurement error models is that the residual distribution is symmetric. Recognizing that we cannot detect skewness we will make this assumption here. In this case the characteristic function of the difference becomes ƒ*(t)² This creates an additional difficulty in that one cannot discern the sign of the residual characteristic function from the characteristic function of the difference. To adjust for this we make the additional assumption that ƒ*(t) is everywhere non-negative. Examples of residual distributions that satisfy the assumptions include the normal, double exponential and Cauchy distributions.

### Estimation of the Residual Characteristic Function

A direct estimate of the characteristic function for the differences is available as, for instance,

$$\begin{matrix}{\hat{f}}_{c}^{*} ( t ) = \frac{1}{nm ( m - 1 )} {\sum }_{i = 1}^{n} {\sum }_{{j}_{1} \neq {j}_{2}}^{} exp [ it ( {y}_{ij1} - {y}_{ij2} ) ] \\ = \frac{2}{nm ( m - 1 )} {\sum }_{i = 1}^{n} {\sum }_{{j}_{1} < {j}_{2}}^{} cos [ t ( {y}_{ij1} - {y}_{ij2} ) ]\end{matrix} \qquad ( 3 )$$

The estimate \hat{ƒ}*ₑ(t) is unbiased but highly variable. Following Zhang (1990) it is valuable to consider a smoothed version of the characteristic function:

\hat{ƒ}*ₛ(t;c)=\hat{ƒ}*ₑ(t)h*(t/c) (4)

where h* is a characteristic function in correspondence with density h. Since h*(t)<1, \hat{ƒ}*ₛ(t;c) is biased downwards. Small values of c tend to give smoother characteristic function estimates. On the other hand as c→∞, \hat{ƒ}*ₛ(t; c)→\hat{ƒ}*ₑ(t). Since the characteristic function is assumed non-negative another reasonable estimate of ƒ*(t) is

$${\hat{f}}_{z}^{*} = \{ \begin{matrix}{\hat{f}}_{e}^{*} ( t ) & if t < z \\ 0 & otherwise\end{matrix} \qquad ( 5 )$$

where Z is the smallest t>0 such that \hat{ƒ}*ₑ(t)=0.

Given a characteristic function estimate \hat{ƒ}*_{d}(t) for the difference εᵢⱼ₁−εᵢⱼ₂, an estimate of the residual characteristic function is \sqrt{[\hat{ƒ}}*ₑ(t)]⁺. A density estimate is obtained by the inversion formula

$$\hat{f} ( x ) = \frac{1}{π} {\int }_{0}^{∞} \sqrt{{[ {\hat{f}}_{z}^{* ( t )} ]}^{+}} cos ( - tx ) ⅆ t \qquad ( 6 )$$

The cumulative distribution function estimate can be obtained by integration of the density estimate. The integration cannot be performed explicitly and must be done numerically.

We will refer to a density or cumulative distribution function estimate based on \sqrt{[\hat{ƒ}}*_{d}(t)]⁺ as a ICF (inverse characteristic function) density or cumulative distribution function estimate. The estimates vary depending upon which estimate for the characteristic function of the differences is used. We refer to the estimate based on (5) as the unsmoothed ICF estimate and an estimate based on (4) as a smoothed ICF estimate.

### Rate of Convergence of the Density Estimates

The use of characteristic functions for the estimation of a density of a random variable Y when Y+X is observed where X is a random variable with known density has been considered by Carroll and Hall (1988) and Zhang (1990). Here we wish to estimate the density of Y−X where Y and X both have the same but unknown density. The problems are similar and we will use a modification of the results of Zhang (1990) to obtain upper bounds for the rate of convergence of the smoothed density estimates.

### Theorem 1 Let \hat{ƒ}(x) be the estimator of ƒ(x) given by (6) with \hat{ƒ}*_{d}(t) given by \hat{ƒ}*(t;cₙ).

Suppose that h* satisfies that

h(x)=h(−x), ∫x²h(x)dx<∞, ∫|xh′(x)|dx<∞, h*(t)=0,∀|t|>1 (7)

Suppose that the constants Cₙ satisfy that

$$\begin{matrix}1 / n \leq {c}_{0} {\vert {\hat{f}}^{*} ( {c}_{n} ) \vert}^{2} / {c}_{n}^{3} , {c}_{0} < ∞ , \vert {f}^{*} ( {c}_{n} ) \vert = {min}_{\vert l \vert \leq {c}_{n}} \vert {f}^{*} ( t ) \vert \\ Then & ( 8 ) \\ {lim}_{n \to ∞} E {‖ \hat{f} - f ‖}^{2} = 0 , ∀ f ∋ ‖ f ‖ < ∞ \\ and & ( 9 ) \\ {sup}_{f : ‖ tf * ( i ) ‖ \leq {M}_{1}} E {‖ \hat{f} - f ‖}^{2} \leq \vert {c}_{0} {C}_{3} + {( {M}_{1} {C}_{1} )}^{2} \vert / 2 π , ∀ n \geq 1. & ( 10 )\end{matrix}$$

An example of a characteristic function satisfying (7) is the function h*(t)that is proportional to the density of the average of four uniform random variables but rescaled so that h*(0)=1. We used this characteristic function for the simulations and examples in later sections. Zhang (1990) shows that the normal, Cauchy, and double exponential distributions satisfy the assumptions of the theorem. The resulting rates of convergence are as follows:

1. Normal: ƒ(x)=exp(−x²/2)/\sqrt{2π}. With cₙ=\sqrt{αlog(n)}, αε(0,1), E∥\hat{ƒ}−ƒ∥²=O([log(n)]⁻¹).

2. Cauchy: ƒ(x)=(1+x²)/π. With cₙ=αlog(n), αε(0,1),

3. Double exponential: ƒ(x)=exp(−|x|)/2. With cₙ=αn^{1/7}, α>0 E∥\hat{ƒ}−ƒ∥²=O(n^{-2/7}).

### The E-M Algorithm for Estimation of the Residual Distribution

As an alternative to the estimation using characteristic functions we consider estimation based upon maximization of a pseudo-loglikelihood

$$E {‖ \hat{f} - f ‖}^{2} = O ( {[ log ( n ) ]}^{- 2} ) . \\ pl ( π ) {\sum }_{i}^{} {\sum }_{j1 \neq j2}^{} log [ {f}_{d} ( {y}_{ij1} - {y}_{ij2} , μ , π , h ) ] \qquad ( 11 )$$

where ƒ_{d} (y, μ,τ, h) is calculated as the density of the difference of two random variables each having density

$$f ( x , μ , π , h ) = {\sum }_{j = 1}^{T} {π}_{j} ϕ ( [ x - {μ}_{j} ] / h ) / h \qquad ( 12 )$$

Here ψ(t)=e^{-t}^{^{2/2}}/\sqrt{2π} and the μⱼ are fixed, equally spaced points symmetrically placed about 0. Let \hat{π} be the maximizer of pl(π). Then ƒ(x ,μ, \hat{π}, h) is used as the estimate of the residual density. We will refer to an estimate that maximizes (11) as a pseudo-likelihood estimator.

The form of the density given in (12) is flexible enough that almost any residual density should be identifiable with large enough T. This method of estimation avoids the numerical integration involved in the characteristic function approach but increases the computational cost by requiring that \hat{π} be calculated as the solution of an optimization problem. Indeed part of the reason for the form of the pseudo-loglikelihood is to simplify the estimation.

### The E-M algorithm

Maximization of pl(π) can be considered as a type of missing data problem and hence the E-M algorithm (Dempster, Laird and Rubin, 1977) can be used. The data points that we observe are the dᵢⱼ₁ⱼ₂:=εᵢⱼ₁−εᵢⱼ₂. These can be thought of as incomplete versions of

εᵢⱼ₁, εᵢⱼ₂, i₁, i₂.

Here i₁ and i₂ are artificial random variables that do not have an explicit role in the algorithm. For each (i,j₁,j₂), the rth value in 1, . . . , T is assigned to i₁ with probability π_{τ} independently of i₂ and εⱼ₂. The conditional distribution of εᵢⱼ₁ given i₁is taken as N (μᵢ₁, h²). The generation of i₂ is defined similarly. The complete data pseudo-loglikelihood is then

$$\frac{1}{{h}^{2}} {\sum }_{i , {j}_{1} , {j}_{2}}^{} log [ {{π}_{i}}_{1} ϕ ( \frac{{ϵ}_{{ij}_{1}} - {μ}_{{j}_{1}}}{h} ) {π}_{{i}_{2}} ϕ ( \frac{{ϵ}_{{ij}_{1}} - {μ}_{{j}_{1}}}{h} ) ] \qquad ( 13 )$$

The details are omitted but the E and M steps of the E-M algorithm can be shown to be: Given current estimates

$${π}_{j}^{( k )} ,$$

$$\begin{matrix}{π}_{j}^{k + 1} α {\sum }_{{ij}_{1} {j}_{2}}^{} [ {p}^{( k1 )} ( j \vert i , {j}_{1} , {j}_{2} ) + {p}^{( k2 )} ( j \vert {ij}_{1} , {j}_{2} ) ] . \\ Here & ( 14 ) \\ {p}^{( k1 )} ( j \vert i , {j}_{1} , {j}_{2} ) ) α {π}_{j}^{( k )} {\sum }_{r}^{} {π}_{r}^{( k )} φ ( \frac{{d}_{{ij}_{1} {j}_{2}} - ( {μ}_{j} - {μ}_{r} )}{h} ) / h \\ and & ( 15 ) \\ {p}^{( k1 )} ( j \vert i , {j}_{1} , {j}_{2} ) ) α {π}_{j}^{( k )} {\sum }_{r}^{} {π}_{r}^{( k )} φ ( \frac{{d}_{{ij}_{1} {j}_{2}} - ( {μ}_{j} - {μ}_{r} )}{h} ) / h & ( 16 )\end{matrix}$$

The constants of proportionality are determined by the constraints that the sums of the

$${π}_{j}^{( k )} ,$$

the

p^{(k1)} (j|i,j₁,j₂) and the p^{k1)} (j|i,j₁,j₂) all equal 1.

### Examples and Application to Expression Data

### A Simulated Data Example

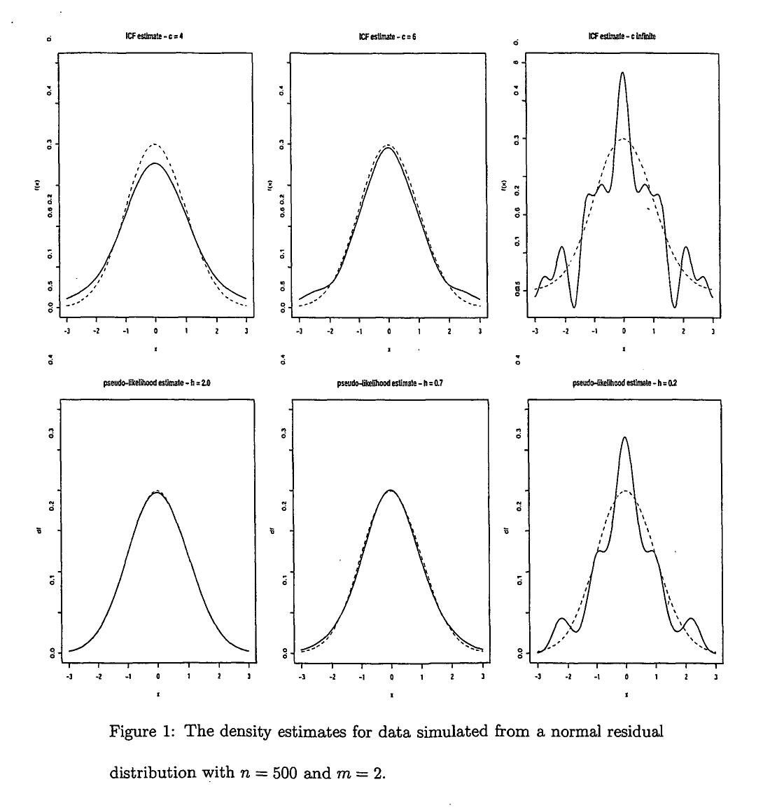

A brief example of the results of estimation when the true density is known is given in FIG. 1. The data in this case were simulated from model (1) with n=500, m=2 and a standard normal residual density. Varying the smoothing parameters h in the case of the pseudo-likelihood estimate and c for the ICF estimate give significantly different estimates. Small values of h allow for more modes in the density estimate and consequently produce more variable estimates than larger values. Similarly small c tend to be associated with smooth density estimates and large c with density estimates with larger numbers of modes.

The smoothed ICF density estimates tend to underestimate the value of the density near 0. This is due to the smoothing factor h*(t/c)<1 in the characteristic function estimates. Smaller values of c are associated with greater bias in this region of the density. The pseudo-likelihood density estimates were better for these data. Generally the pseudo-likelihood estimates can be expected to perform well when the residual distribution is close to normal since the normal density is used as the kernel in (12).

The density estimates in FIG. 1 are symmetric. Generally this will always be the case: The ICF estimates are symmetric since both the negative and positive differences yᵢ₁−yᵢ₂ and yᵢ₂−yᵢ₁ are included in the construction of (3) resulting in symmetric characteristic function estimates for (4) and (5). For the pseudo-likelihood estimates it can be shown that if the μ are chosen to be symmetric about 0 and the initial weight πⱼ⁽⁰⁾ for μⱼ is the same as the initial weight μⱼ in (14), then the final density estimate will be symmetric.

### The Smoothing Parameters

The density estimates usually vary significantly with different smoothing parameters. The procedures for the selection of smoothing parameters discussed here were used for the expression data in the following sections relating to gene expression and simulations.

The multiplication of the characteristic function estimate (3) by h*(t/c) implies that the resultant characteristic function estimate will be 0 for |t|>c. Consequently a reasonable upper bound for the appropriate smoothing parameter c is Z, the smallest t>0 such that \hat{ƒ}ₑ(t)=0. In our experience (see the simulations) we have found that even with values of c as large as Z there is significant bias in the distribution function estimates for the sample sizes of primary interest (n≦00). For this reason we also consider the unsmoothed ICF density estimate.

For the pseudo-likelihood estimates we determine h using the l_{∞} distance between (i) the unbiased estimate (3) of the distribution for the difference between two residuals and (ii) the cumulative distribution of the difference of two random variables resulting from the residual density estimate (12) for the h under consideration. Since the variance for a random variable from (12) is at least h², a reasonable upper bound h₀² for the smoothing parameter is the sample variance of the differences. We select a smoothing parameter ĥ as the first h in {α^{k}h₀: 0<α<1} such that the l_{∞} distance for α^{k+1} h₀ is greater than the l_{∞} for α^{k}.

### Gene Expression Data

We illustrate the estimation of the residual distribution with the estimates obtained for gene expression data from brain tissue. The data are available at

http://idefix.upr420.vjfcnrs.fr/hgi-bin/exgenx.sh?CLNINDEXx.html The expression values for brain tissue for n=7483 genetic sequence tags were obtained as described in Piétu et. al., (1996). There were m=2 repeated measurements for each sequence tag.

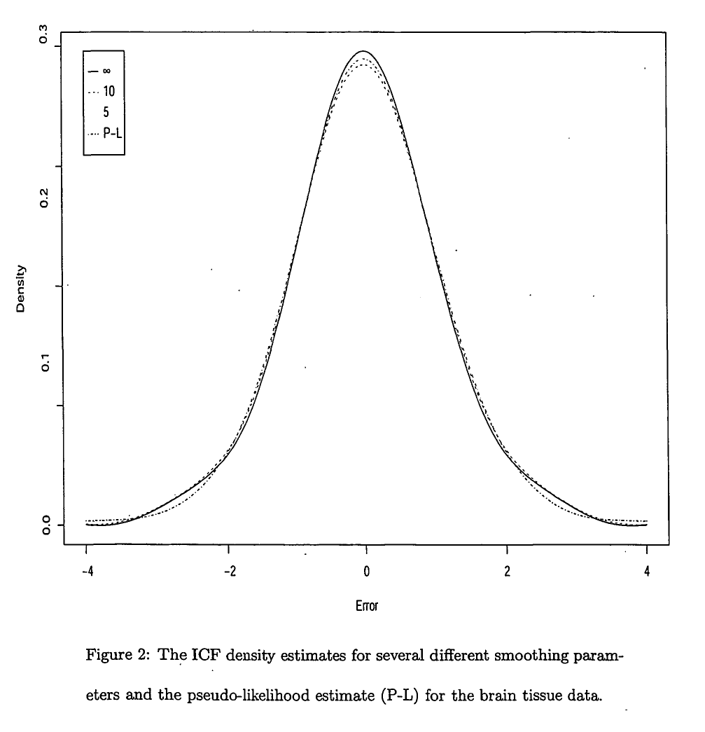

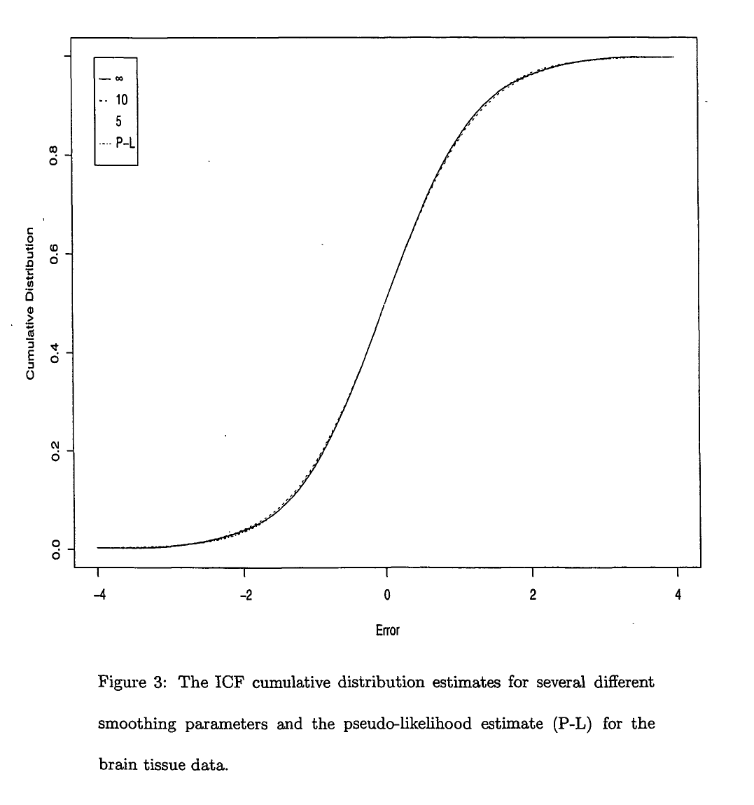

Plots of ICF densities with various smoothing parameters (c=∞ gives the unsmoothed estimate) are given in FIG. 2. The density estimates are all very similar in this case. More important for calculating confidence intervals are the cumulative distributions which are given in FIG. 3.

The 95\% confidence intervals for the differences μ₁ᵢ−μ₂ᵢ described previously would be obtained as

\overline{y}_{1i·}−\overline{y}_{2i·}±π\sqrt{σ₁²/m+σ₂²/m}

where τ is the 0.975th quantile of the residual distribution. The estimates of τ for the ICF estimate of the residual distribution with c=5, with no smoothing and pseudo-likelihood estimate are 2.37, 2.27 and 2.21 respectively. Thus one would construct a 95\% confidence interval from the unsmoothed ICF estimate as

\overline{y}_{1i·}−\overline{y}_{2i·}±2.27\sqrt{σ₁²/m+σ₂²/m}

which would be larger than the conventional normal based interval:

\overline{y}_{1i·}−\overline{y}_{2i·}±1.96\sqrt{σ₁²/m+σ₂²/m}

### Simulation Results

To further evaluate the methodologies several simulations were considered. For each set of simulations, samples from (1) were generated from a given residual distribution with n=500 and m=2. The residual distributions considered were the normal, double exponential and Cauchy distributions. The estimators considered were (i) the unsmoothed ICF estimate resulting from (5) (ii) the smoothed ICF estimate resulting from (4) with c taken as Z, the smallest t>0 such that \hat{ƒ}ₑ(t)=0 , and (iii) the pseudo-likelihood estimate with the smoothing parameter h chosen using the l_{∞} criterion, discussed previously, with α=0.8. For (i) and (ii) 10000 simulated samples were drawn. For (iii) the first 1000 samples were used. A summary of the results of the simulations are given in Tables 1-2. The estimates of the probabilities from (ii) are biased downwards. In contrast the estimates of the probabilities and the quantiles from (i) and (iii) are quite reasonable for these samples sizes.

The methodologies discussed in this article provide a means of estimating the residual distribution for models of the form (1). Such models arise in data settings, such as the analysis of gene expression data, where there are a large number of comparisons or mean estimations with a similar measurement error process. The purposes of obtaining density estimates may vary. One could use them directly to adjust confidence intervals or to check a parametric residual distribution assumption.

Theorem 1 indicates that the ICF estimates provide for consistent residual distribution estimation. While the upper bounds on the rates of convergence given above suggest that a large number of observations are required for consistent estimation of the density function, the simulation results indicate that reasonable estimates of the cumulative distribution probability estimates can be obtained with n≧500, which is usually the situation for gene expression data. The simulation results further favor less smoothing than one might expect. The pseudo-likelihood density estimates give reasonable density estimates as well. In contrast to the characteristic function based estimates however, more computational power is required to obtain them.

It should be appreciated that the outcome of the process of the invention can be applied to the original data set or array or it may be applied to a new one. Moreover, the process may be applied in three different ways:

1. It can be used to determine the reliability of differences across two different samples (obtained, say, from two different tissues), i.e. different outcomes of a physical measurement. This can be done with the original data set or array on which the process was applied. Since the original data set has repeated measurements, the process would typically be applied to the mean of the repeated measurements. It can also be applied to a new data set. The new data set may have only one measurement. Or in the case of repeated measurements in the new data set, the outcome of the original data set can be applied to the mean of the measurements or of course the process may be repeated with the new data set.

2. It can also be used to determine if a measured value deviates from all of the other measured values in the distribution. This is not the same as point 1. Here the comparison is not between two measured values but rather between one measured value and all of the others in a distribution. The idea here is that the measured values' “place” in the distribution is assessed relative to a threshold established by the random error estimation process. If the measured value exceeds the threshold, it is then said to represent a different physical measurement relative to the other values in the distribution. For example, most genes in an array may not be expressed above the background noise of the system. These genes would form the major portion of the distribution. Other genes may lie outside of this distribution as indicated by their values exceeding a threshold determined by the random error estimation. These genes would be judged to represent a different physical process.

3. The process may also be used to establish “outlier” values. In the preceding description, they are also described as “an extreme value in a distribution of values.” Outlier data often result from uncorrectable measurement errors and are typically deleted from further statistical analysis.” Point 2, above, also refers to detecting an extreme value but in that case the extreme value is based on the intensity of the measurement. That is not an outlier as intended here. Here, outlier refers to an extreme residual value. An extreme residual value often reflects an uncorrectable measurement error.

Although preferred forms of the invention have been disclosed for illustrative purposes, those skilled in the art will appreciate that many additions, modifications and substitutions are possible without departing from the scope and spirit of the invention as defined by the accompanying claims.

TABLE 1
The estimated mean F(qₚ) with n = 500, m = 2 based on simu-
lation. Here qₚ is the pth quantile for the generating residual distribution.
Method (i) is the unsmoothed characteristic function based estimate (ii) the
smoothed estimate with c = 5.0 and (iii) the E-M based estimate. Estimated
standard deviations are given in parentheses.

| | | p | | | |
|---|---|---|---|---|---|
| Distribution | Method | 0.75 | 0.9 | 0.95 | 0.975 |
| Normal | (i) | 0.75 (0.02) | 0.89 (0.01) | 0.95 (0.01) | 0.98 (0.01) |
| | (ii) | 0.66 (0.02) | 0.78 (0.02) | 0.83 (0.02) | 0.88 (0.02) |
| | (iii) | 0.75 (0.01) | 0.9 (0.01) | 0.95 (0.01) | 0.97 (0.01) |
| Double | (i) | 0.75 (0.02) | 0.9 (0.01) | 0.95 (0.01) | 0.97 (0.01) |
| Exponential | (ii) | 0.64 (0.02) | 0.79 (0.01) | 0.87 (0.01) | 0.92 (0.01) |
| | (iii) | 0.74 (0.02) | 0.9 (0.01) | 0.95 (0.01) | 0.97 (0.01) |
| Cauchy | (i) | 0.74 (0.02) | 0.9 (0.01) | 0.95 (0.01) | 0.98 (0.00) |
| | (ii) | 0.62 (0.01) | 0.8 (0.01) | 0.9 (0.01) | 0.95 (0.01) |
| | (iii) | 0.72 (0.06) | 0.88 (0.07) | 0.94 (0.05) | 0.97 (0.03) |

TABLE 2
The estimated mean pth quantile with n = 500, m = 2 based
on simulation. Method (i) is the unsmoothed characteristic function based
estimate (iii) the pseudo-likelihood estimate. Estimated standard deviations
are given in parentheses.

| | | p | | | |
|---|---|---|---|---|---|
| Residual Distribution | Method | 0.75 | 0.9 | 0.95 | 0.975 |
| Normal | Actual | 0.67 | 1.28 | 1.64 | 1.96 |
| | (i) | 0.66 (0.07) | 1.32 (0.08) | 1.68 (0.10) | 1.97 (0.15) |
| | (iii) | 0.66 (0.05) | 1.28 (0.05) | 1.66 (0.07) | 1.99 (0.09) |
| Double Exponential | Actual | 0.69 | 1.61 | 2.30 | 3.00 |
| | (i) | 0.7 (0.08) | 1.6 (0.12) | 2.29 (0.2) | 3.01 (0.32) |
| | (iii) | 0.73 (0.08) | 1.58 (0.11) | 2.29 (0.18) | 3.2 (0.28) |
| Cauchy | Actual | 1.00 | 3.08 | 6.31 | 12.71 |
| | (i) | 1.04 (0.11) | 3.09 (0.39) | 6.28 (0.83) | 12.4 (2.01) |
| | (iii) | 1.44 (1.18) | 3.7 (2.02) | 7.3 (2.31) | 14.74 (3.7) |

### References

Carrol, R. J. and Hall, P. (1988). “Optimal Rates of Convergence for Deconvolving a Density”, Journal of the American Statistical Association, 83, 1184-1186.

Chen, Dougherty, & Bittner, (1997). “Ratio-based Decisions and the Quantitative Aanalysis of cDNA Microarray Images”, Journal of Bionmedical Optics, 2, 364-374. Dempster, A. P., Laird, N. M. and Rubin, D. B., (1977). “Maximum Likelihood from Incomplete Data via the E-M Algorithm”, Journal of the Royal Statistical Society, Series B, 39, 1-38.

McLachlan, G. and Krishnan, T. (1997). {\it The EM Algorithm and Extensions}, Wiley, N.Y.

Piétu, G, Alibert, O., Guichard, V., Lamy, B., Bois, F., Leroy, E., Mariage-Smason, R., Houlgatte, R., Soularue, P. and Auffray, C. (1996). “Novel Gene Transcripts Preferentially Expressed in Human Muscles Revealed by Quantitative Hybridization of a High Density cDNA Array”, Geiiome Research, 6, 492-503.

Zhang, C. (1990). “Fourier Methods for Estimating Mixing Densities and Distributions”, Annals of Statistics}, 18, 806-831.

The disclosures of the preceding references are incorporated herein in their entirty.

What is claimed is:

1. A method for improving the reliability of physical measurements obtained from array hybridization studies performed on an array having a large number of genomic samples including a replicate subset containing a small number of replicates insufficient for making precise and valid statistical inferences, comprising the step of estimating an error in measurement of a sample by combining estimates obtained with individual samples in the replicate subset, and utilizing the estimated sample error as a standard for accepting or rejecting the measurement of a sample under test.

2. The method of claim 1 wherein the combining step includes taking the difference between estimates obtained for a pair of samples in the replicate subset.

3. The method of claim 2 wherein the difference is taken between the estimates for all permutations of pairs of samples for the replicate subset.

4. The method of claim 3 wherein the difference is taken between the estimates for all combinations of pairs of samples for the replicate subset.

5. The method of any one of claims 1-4 used with respect to two new samples to establish a confidence level that two samples under test express different outcomes of a physical measurement.

6. The method of any one of claims 1-4 wherein the estimates of measurement error are used to plan, manage and control array hybridization studied on the basis of (a) the probability of detecting a true difference of specified magnitude between physical measurements of a given number of samples under test, or (b) the number of samples under test required to detect a true difference of specified magnitude.

7. The method of anyone of claims 1-6 wherein the sample under test is in the array.

8. The method of anyone of claims 1-6 wherein the sample under test is in an array other than the array.

9. The method of anyone of claims 1-6 used to determine whether the sample under test deviates substantially from all of the other values in a selected portion of an array.

10. The method of any preceding claim wherein there are no replicates corresponding to the sample under test.
